(19) United States
(12) Patent Application Publication
(10) Pub. No.: US 2019/0365263 A1
(43) Pub. Date: Dec. 5, 2019

(54) DIGITAL STETHOSCOPE USING MECHANO-ACOUSTIC SENSOR SUITE

(71) Applicant: MC10, Inc., Lexington, MA (US)

(72) Inventors: Milan Raj, Natick, MA (US); Roozbeh Ghaffari, Cambridge, MA (US); Bryan McGrane, Cambridge, MA (US); Brandon Suleski, Chelmsford, MA (US)

(21) Appl. No.: 16/478,798

(22) PCT Filed: Jan. 17, 2018
(86) PCT No.: PCT/US18/13966

§ 371 Date: Jul. 17, 2019

Related U.S. Application Data
(60) Provisional application No. 62/447,684, filed on Jan. 18, 2017.

(51) Int. Cl. A61B 5/0402; A61B 5/00; A61B 5/024; A61B 5/11; A61B 7/04

(52) U.S. Cl. CPC A61B 5/0402; A61B 5/0024; A61B 5/0017; A61B 5/01; A61B 5/11; A61B 5/6833; A61B 7/04; A61B 5/024

(57) ABSTRACT

A system and method for sensing acoustic data generated by a user is disclosed. The system includes a wearable sensor including an accelerometer sensor in contact with the skin of the patient to measure mechano-acoustic signals generated from a bodily function and generate an accelerometer waveform. A controller receives the accelerometer waveform from the accelerometer sensor to determine a measurement of the bodily function. The wearable sensor includes features to directly contact the skin and isolate the accelerometer sensor to produce more accurate output signals.

### CROSS-REFERENCE TO RELATED APPLICATIONS

The present application claims the benefit of and priority to U.S. Provisional Patent Application No. 62/447,684, filed Jan. 18, 2017, entitled, “Digital Stethoscope Using Mechano-Acoustic Sensor Suite,” which is hereby incorporated by and referenced herein in its entirety.

### TECHNICAL FIELD

The present disclosure relates generally to mechano-acoustical body sensors. More particularly, aspects of this disclosure relate to using wearable mechano-acoustic sensors to measure acoustic signals from a body.

### BACKGROUND

Integrated circuits (ICs) are the cornerstone of the information age and the foundation of today's information technology industries. The integrated circuit, a.k.a. “chip” or “microchip,” is a set of interconnected electronic components, such as transistors, capacitors, and resistors, which are etched or imprinted onto a semiconducting material, such as silicon or germanium. Integrated circuits take on various forms including, as some non-limiting examples, microprocessors, amplifiers, flash memories, application specific integrated circuits (ASICs), static random access memories (SRAMs), digital signal processors (DSPs), dynamic random access memories (DRAMs), erasable programmable read only memories (EPROMs), and programmable logic. Integrated circuits are used in innumerable products, including computers (e.g., personal, laptop, and tablet computers), smartphones, flat-screen televisions, medical instruments, telecommunication and networking equipment, airplanes, watercraft, and automobiles.

Advances in integrated circuit technology and microchip manufacturing have led to a steady decrease in chip size and an increase in circuit density and circuit performance. The scale of semiconductor integration has advanced to the point where a single semiconductor chip can hold tens of millions to over a billion devices in a space smaller than a U.S. penny. Moreover, the width of each conducting line in a modern microchip can be made as small as a fraction of a nanometer. The operating speed and overall performance of a semiconductor chip (e.g., clock speed and signal net switching speeds) has concomitantly increased with the level of integration. To keep pace with increases in on-chip circuit switching frequency and circuit density, semiconductor packages currently offer higher pin counts, greater power dissipation, more protection, and higher speeds than packages of just a few years ago.

The advances in integrated circuits have led to related advances within other fields. One such field is sensors for monitoring body readings such as temperature, blood pressure, heart rate, and the like. Advances in integrated circuits have allowed sensors to become smaller and more efficient, while simultaneously becoming more capable of performing complex operations. Other advances in the field of sensors and circuitry in general have led to wearable circuitry, a.k.a. “wearable devices” or “wearable systems.” Within the medical field, as an example, wearable devices have given rise to new methods of acquiring, analyzing, and diagnosing medical issues with patients, by having the patient wear a sensor that monitors specific characteristics. Related to the medical field, other wearable devices have been created within the sports and recreational fields for the purpose of monitoring physical activity and fitness. For example, a user may don a wearable device, such as a wearable running coach, to measure the distance traveled during an activity (e.g., running, walking, etc.), and measure the kinematics of the user's motion during the activity.

### SUMMARY

Certain bodily functions may be monitored by analyzing sounds from the heart, lungs, and intestines. Such acoustic data may assist in diagnosis of abnormalities in the respiratory system, circulatory system, or digestion system, among others. One well-known instrument used by physicians is a manual stethoscope that a medical practitioner uses to listen to sounds generated by the respiratory system, circulatory system, or digestion system in a patient. However, a manual stethoscope is not sensitive to a full range of sounds and requires human interpretation of the sounds. Further a manual stethoscope is not capable of discerning other useful sound signals that may not be detectable by the human ear.

Recently, electrical acoustic sensors have made the functions of a traditional stethoscope possible in an electronic stethoscope that provides amplification of detected sounds so that it is easier to detect heart and lung sounds. However, traditional electronics with rigid packaging cannot measure mechanical vibrations with sufficient sensitivity due to lack of direct mechanical coupling to skin. Further, since such instruments are generally not wearable, they cannot provide continuous monitoring of a patient. To the extent that acoustical sensing has been used in a wearable device, an accelerometer sensor has been used for sensing mechano-acoustical signals. However, the internal components of such devices may impede the accurate determination of acoustic signals from a patient due to dampening. Without unique design and positioning of the accelerometer sensor in the sensor configuration, and verification with a heartbeat such as an ECG signal, useful fine signals that may be real physiological signals cannot be used.

Thus, there is a need for an accurate acoustic system to determine acoustic data from a patient. There is a further need for a wearable sensor that allows the continuous sensing of acoustic signals from a patient. There is also a need for an accurate wearable acoustic sensor where an accelerometer is configured on the sensor housing that minimizes interference.

One disclosed example is a sensor system for sensing sound associated with a bodily function of a user. The system includes a wearable sensor including a planar mechano-acoustic conductor in direct contact with the skin of the user to measure mechano-acoustic vibration signals generated from a bodily function and generate a vibration waveform. A controller receives the mechano-acoustic vibration waveform from the wearable sensor to determine a measurement of the bodily function.

Another example is a wearable sensor for detecting a mechano-accoustical signal from a user. The sensor includes a rectangular planar body composed of encapsulation material. A first island is located in the middle of the rectangular planar body. A second island includes an accelerometer. The second island is isolated from the first island using flexible interconnections to buffer vibrations. The second island is located in proximity to a corner of the rectangular planar body.

Another example is a method of detecting an acoustic signal from a user. A wearable sensor including a planar mechano-acoustic conductor is attached in direct contact with the skin of the user to measure mechano-acoustic vibration signals generated from a bodily function and generate a vibration waveform. A measurement of the bodily function is determined from the mechano-acoustic vibration waveform via a controller.

The above summary is not intended to represent each embodiment or every aspect of the present disclosure. Rather, the foregoing summary merely provides an exemplification of some of the novel aspects and features set forth herein. The above features and advantages, and other features and advantages of the present disclosure, will be readily apparent from the following detailed description of representative embodiments and modes for carrying out the present invention when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a system of wearable sensors functioning as a digital stethoscope for detecting and characterizing acoustic signals from a user;

FIG. 2 is a block diagram of one of the wearable sensor devices in FIG. 1;

FIG. 3A-3D are graphs showing sampled ECG and accelerometer signals from the sensor devices in FIG. 1;

FIG. 4A is a top view of one of the sensors in FIG. 1;

FIG. 4B is a perspective view of one of the sensors in FIG. 1;

FIG. 4C is a side view of one of the sensors in FIG. 1 worn by the user; and

FIG. 5 is a flow diagram showing the process of measuring and recording mechano-acoustic data associated with the sensors in the system in FIG. 1 for monitoring the circulatory system in the user.

The present disclosure is susceptible to various modifications and alternative forms, and some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. There are shown in the drawings, and will herein be described in detailed, representative embodiments with the understanding that the present disclosure is to be considered as an exemplification or illustration of the principles of the present disclosure and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed: the singular includes the plural and vice versa; and the word “including” means “including without limitation.” Moreover, words of approximation, such as “about,” “almost,” “substantially,” “approximately,” and the like, can be used herein in the sense of “at, near, or nearly at,” or “within 3-5% of,” or “within acceptable manufacturing tolerances,” or any logical combination thereof, for example.

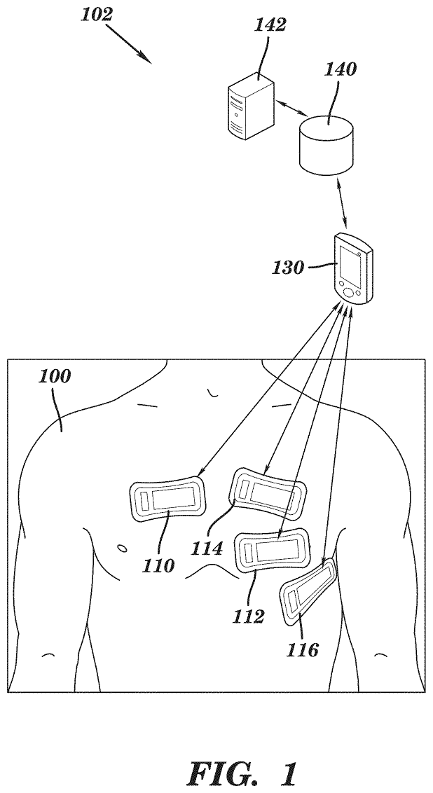

FIG. 1 shows a monitoring system 102 that can be employed by a user 100 for monitoring of acoustic data such as heartbeat or blood circulation sounds. The system 102 can include multiple wearable sensor devices 110, 112, 114, and 116. Each of the wearable sensor devices 110, 112, 114, and 116 can include an accelerometer that can detect the motion and vibrations transmitted to the skin of the user 100 produced by the organs of the body, such as the heart and circulatory system in this example. The wearable sensor devices 110, 112, 114, and 116 may also function as a heartbeat sensor that can, for example, obtain an electro-cardiogram (ECG) signal, a seismocardiogram (SCG) waveform, or a PPG signal indicative of the heartbeat.

In this example, the sensors 110, 112, 114, and 116 are attached to the skin at various locations on the body to efficiently obtain acoustic data relating to the function of the heart of the user 100. Thus the wearable sensor device 110 is preferably positioned on the chest in the position shown in FIG. 1 in proximity to the aortic valve of the heart. The wearable sensor device 112 is preferably positioned on the chest in the position shown in FIG. 1 near the transcuspid value of the heart. The wearable sensor device 114 is preferably positioned on the chest in the position shown in FIG. 1 near the pulmonary valve of the heart. The wearable sensor device 116 is preferably positioned on an area of the chest near the mitral valve of the heart. However, as will be explained below, the sensor devices such as the sensor device 110 can be located in any area relative to the source of desired acoustic signals, such as in proximity to the lungs to monitor respiratory functions or the intestines to monitor digestion functions. Of course less than four or more than four of wearable sensor devices such as the devices 110, 112, 114, and 116 can be used depending on the desired acoustic data.

The sensor device 110 produces an output signal that is based on sampling of accelerometer signals indicative of mechano-acoustic motion and vibration generated by heart activity (e.g., blood flow between heart chambers) from the aortic valve. The wearable sensor device 110 can also produce other output signals (e.g., an ECG or similar signal) that is based on sampling ECG electrodes or other inputs. Similarly, in this example, the other sensor devices 112, 114, and 116 also produce an output signal that is based on sampling of accelerometer signals from mechano-acoustic motion and vibration generated by blood flow through their corresponding valves. Of course other acoustic data may be detected by attaching another sensor or moving one of the sensor devices 110, 112, 114, and 116 to another location on the user 100. For example, respiratory monitoring can be performed by the system 102 by sampling accelerometer signals from mechano-acoustic motion and vibration generated by airflow (e.g., expansion and contraction of the airway and sound and/or vibrations resulting from airflow passing through an airway).

The wearable sensor devices 110, 112, 114, and 116 can be in communication with a smart device or hub such as a user device 130. The user device 130 can be a computing device such as a smart phone, a tablet, a laptop or desktop computer, a personal digital assistant, or a network of computers (e.g., a cloud or a cluster). The user device 130 can be used to control, configure, and/or program the wearable sensor devices 110, 112, 114, and 116. For example, the user device 130 can configure the wearable sensor devices to sense certain audio signals related to a particular function such as heart monitoring. Identification and location information may also be set for each of the wearable sensor devices by the user device 130 for the particular function. Although the wearable sensor devices 110, 112, 114, and 116, as described herein, are used for non-invasive acoustic sensing for bodily functions such as respiratory and/or heart monitoring, each can have other measurement and sensing functions in relation to the user 100.

The acoustic data from the wearable sensor devices 110, 112, 114, and 116 representative of heart activity and, optionally, the data from the ECG sensor representative of the heartbeat signal can be uploaded to a cloud storage server 140 periodically (e.g., in time-stamped blocks) or continuously (e.g., streamed) and analyzed by applications running on one or more cloud application servers 142 from the sensor devices directly or via the user device 130. The data can be processed in real time or using post-processing techniques. The user can access the data, the analysis applications or the output of the applications by accessing the cloud server 142, such as through a website.

As will be explained below, any of the sensors 110, 112, 114, and 116 may be used to sense and store accelerometer data representative of sensed acoustical data and ECG or other heartbeat generated data. As will be explained below, the user device 130 can include software that processes the sensed data in order to determine the occurrence and characterization of conditions such as abnormal heart operation, respiratory abnormalities, digestive abnormalities, etc. Alternatively, one or more cloud applications executed on the cloud application server 160 can process the data received from the sensors 110, 112, 114, and 116 (e.g., via the user device 130) to the determination of the occurrence and characterization of detected abnormalities based on the sensed acoustic data.

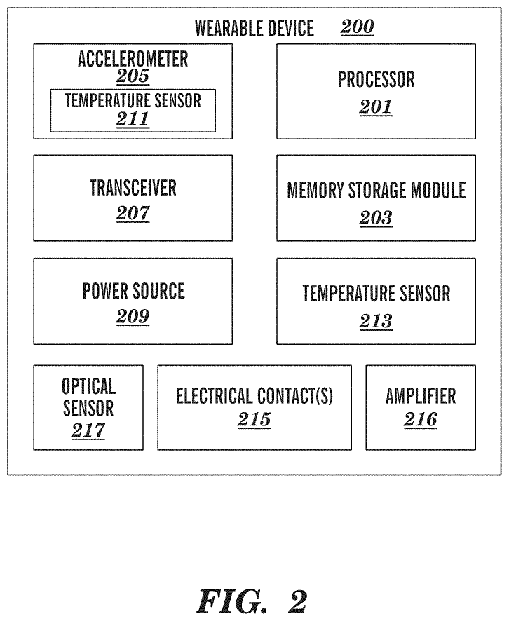

FIG. 2 shows a diagrammatic example of a wearable sensor device 200 such as the sensor devices 110, 112, 114, and 116 in FIG. 1 in accord with aspects of the present disclosure. The wearable device 200 can provide conformal sensing capabilities, providing mechanically transparent close contact with a surface (such as the skin or other portion of the body) to provide measurement and/or analysis of physiological information from the user 100. According to some embodiments, the wearable device 200 senses, measures, or otherwise quantifies the mechano-acoustic signals of at least one body part of a user upon which the wearable device 200 is located. Additionally, or in the alternative, according to some embodiments, the wearable device 200 senses, measures, or otherwise quantifies the temperature of the environment of the wearable device 200, including, for example, the skin and/or body temperature at the location that the wearable device 200 is coupled to the body of a user. Additionally, or in the alternative, according to some embodiments, the wearable device 200 senses, measures, or otherwise quantifies other characteristics and/or parameters of the body (e.g., human or animal body) and/or surface of the body, including, for example, temperature, motion, electrical signals associated with cardiac activity (e.g., ECG), electrical signals associated with muscle activity (e.g., electromyography (EMG)), changes in electrical potential and impedance associated with changes to the skin (e.g., galvanic skin response), electrical signals of the brain (e.g., electroencephalogram (EEG)), bioimpedance monitoring (e.g., body-mass index, stress characterization, and sweat quantification), and optically modulated sensing (e.g., photoplethysmography (PPG) and pulse-wave velocity), and the like.

The wearable device 200 described herein can be formed as a patch. The patch can be flexible and stretchable, and can include stretchable and/or conformal electronics and/or conformal electrodes disposed in or on a flexible and/or stretchable substrate. Alternatively, the wearable device 200 can be rigid but otherwise attachable to a user. In accordance with some embodiments of the invention, the wearable device 200 can include portions that are stretchable and/or conformable and portions that are rigid. Thus, the wearable device 200 can be any device that is wearable on a user, such as coupled to the skin of the user, to provide measurement and/or analysis of physiological information of the user. For example, the wearable device can be adhered to the body by adhesive (e.g., a pressure sensitive adhesive), held in place against the body by tape or straps, or held in place against the body by clothing. The more conformal the sensing device, the more likely it is to stay in position on the skin and produce more reliable and accurate sensor data.

In general, the wearable device 200 of FIG. 2 can include at least one processor 201 connected to one or more associated memory storage modules 203. The wearable device 200 can further include one or more sensors, such as an accelerometer 205 and/or a temperature sensor 213 and/or an optical sensor 217, connected to the processor 201. The wearable device 200 can optionally include one or more wireless transceivers, such as transceiver 207, connected to processor 201 for communicating with other sensor devices such as the sensor devices 110 and 112 or other computing devices such as the user device 130 in FIG. 1. The wearable device 200 can also include a power source 209 connected to the components of the wearable device 200 to power the processor 201, the memory 203, and each of the other components of the wearable device 200. In accordance with some embodiments, the wearable device 200 can be configured to draw power from a wireless connection or an electromagnetic field (e.g., an induction coil, an NFC reader device, microwaves, and light). The wearable device can include, for example, an induction coil and a wireless charging circuit that produces electric power when exposed to an electric or magnetic field to charge the battery and provide power to the wearable device.

The processor 201 can be used as a controller that is configured to control the wearable device 200 and components thereof based on computer program code (e.g., one or more software modules). Thus, the processor 201 can control the wearable device 200 to receive and store sensor data from one or more of the sensors 205, 213, 217. The sensor data can be calibrated and used to determine measures indicative of temperature, motion, and/or other physiological data (e.g., ECG, EMG, EEG signals and data), and/or analyze such data indicative of temperature, motion, and/or other physiological data according to the principles described herein.

The memory storage module 203 can be configured to save the generated sensor data (e.g., the time when a pulse in blood flow is sensed, accelerometer 205 information, temperature sensor 213 information, or other physiological information, such as ECG, EMG, EEG signals and data) or information representative of acceleration and/or temperature and/or other physiological information derived from the sensor data. Further, according to some embodiments, the memory storage module 203 can be configured to store the computer program code that controls the processor 201. In some implementations, the memory storage module 203 can include volatile and/or non-volatile memory. For example, the memory storage module 203 can include dynamic memory, flash memory, static memory, solid state memory, removable memory cards, or any combination thereof. In certain examples, one or more of the memory storage modules 203 can be removable from the wearable device 200. In some implementations, one or more of the memory storage modules 203 can be local to the wearable device 200, while in other examples one or more of the memory storage modules 203 can be remote from the wearable device 200. For example, one or more of the memory storage modules 203 can include the internal memory of a smartphone such as the user device 130 in FIG. 1 that is connected by a wired or wireless connection to the wearable device 200, such as through radio frequency communication protocols including, for example, WiFi, Zigbee, Bluetooth®, medical telemetry, and near-field communication (NFC), and/or optically using, for example, infrared or non-infrared LEDs. In such an example, the wearable device 200 can optionally communicate (e.g., wirelessly) with the user device 130 via an application (e.g., program) executing on the user device 130.

In some embodiments, the generated data, including the temperature information, the acceleration information, and/or the other physiological information (e.g., ECG, EMG, EEG etc.), can be stored in one or more of the memory storage modules 203 for processing at a later time. Thus, in some embodiments, the wearable device 200 can include more than one memory storage module 203, such as one volatile and one non-volatile memory storage module 203. In other examples, the memory storage module 203 can store the information indicative of motion (e.g., acceleration information), temperature information, physiological data, or analysis of such information indicative of motion, temperature, and physiological data according to the principles described herein, such as storing historical acceleration information, historical temperature information, historical extracted features, and/or historical locations. The memory storage module 203 can also store time and/or date information about when the information was received from the sensor. For example, each data element or block of data elements can be associated with a date and/or time at which it was created.

Although described as the processor 201 being configured according to computer program code in the form of software and firmware, the functionality of the wearable device 200 can be implemented based on hardware, software, or firmware or a combination thereof. For example, the memory storage module 203 can include computer program code in the form of software or firmware that can be retrieved and executed by the processor 201. The processor 201 executes the computer program code that implements the functionality discussed below with respect to determining the on-body status of the wearable device 200, the location of the wearable device 200 on a user, and configuring functionality of the wearable device 200 (e.g., based on the on-body status and sensed location). Alternatively, one or more other components of the wearable device 200 can be hardwired to perform some or all of the functionality.

The power source 209 can be any type of rechargeable (or single use) power source for an electronic device, such as, but not limited to, one or more electrochemical cells or batteries, one or more photovoltaic cells, or a combination thereof. In the case of the photovoltaic cells, the cells can charge one or more electrochemical cells and/or batteries. In accordance with some embodiments, the power source 209 can be a small battery or capacitor that stores enough energy for the device to power up and execute a predefined program sequence before running out of energy, for example, an NFC-based sensing device.

As discussed above, the wearable device 200 can include one or more sensors, such as the accelerometer 205, a temperature sensor 213, electrical contacts 215 (e.g., electrical contacts or electrodes), and/or an optical sensor 217. In accordance with some embodiments, one or more of the sensors, such as accelerometer 205, the optical sensor 217, and/or electrical contacts 215, can be separate components from the wearable device 200. That is, the wearable device 200 can be connected (by wire or wirelessly) to each sensor (e.g., accelerometer 205, temperature sensor 213, electrical contacts 215, and optical sensor 217). This enables the wearable device 200 to sense conditions at one or more locations that are remote from the wearable device 200. In accordance with some embodiments, the wearable device 200 can include one or more integral sensors in addition to one or more remote sensors.

The accelerometer 205 measures and/or generates acceleration information indicative of a motion and/or acceleration of the wearable device 200, including information indicative of a user wearing, and/or body parts of the user wearing, the wearable device 200. In accordance with one embodiment, the accelerometer 205 within the wearable device 200 can include a 3-axis accelerometer that generates acceleration information with respect to the x-axis, the y-axis, and the z-axis of the accelerometer based on the acceleration experienced by the wearable device 200. Alternatively, the wearable device 200 can include three independent accelerometers (not shown for illustrative convenience) that each generate acceleration information with respect to a single axis, such as the x-axis, the y-axis, or the z-axis of the wearable device 200. Alternatively, the wearable device 200 can include an inertial measurement unit (IMU) that measures the angular velocity, the orientation, and the acceleration using a combination of one or more accelerometers, gyroscopes, and magnetometers. Thus, although generally referred to herein as an accelerometer 205, the accelerometer 205 can be any motion sensing element or combination of elements that provides acceleration information. In this example, the accelerometer may be specialized to detect mechano-acoustic vibrations. Of course other acoustic sensors such as a microelectromechanical system (MEMs) microphone may be used. A MEMs microphone will transduce the pressure waves propagating from the skin, generated by mechanical vibrations from within the body, into electrical signals that processor 201 can divert to memory storage module 203 or transceiver 207.

In this example, the accelerometer 205 is an MPU-6500 manufactured by Invensense. According to some embodiments, the accelerometer 205 includes a detection range of ±2 times the force of gravity (Gs). However, the range can vary, such as being ±16 Gs or ±2 Gs. Further, the accelerometer 205 can have a sampling rate of 100 hertz (Hz) such that each second the accelerometer 205 generates 300 points of acceleration information, or 100 points within each axis. However, the sampling rate can vary, such as being 20 Hz to 500 Hz.

According to some embodiments, one or more sensors of the wearable device 200, such as the accelerometer 205, can include a built-in temperature sensor, such as the temperature sensor 211 within the accelerometer 205. For example, the temperature sensor 211 within the accelerometer 205 can be used to calibrate the accelerometer 205 over a wide temperature range and to measure the temperature of the area of the body that the accelerometer 205 is coupled to. Other temperature sensors included with other device components can also be used. Other than the accelerometer 205, and temperature sensor 211, other subcomponents or elements of the wearable device 200 can include one or more microelectromechanical system (MEMS) components within the wearable device 200 that is designed to measure motion or orientation (e.g., angular-rate gyroscope, etc.).

In accordance with some embodiments of the invention, an accelerometer (or an acoustic sensor) such as the accelerometer 205 of the wearable sensor device 200 shown in FIG. 2 can be used to detect and measure a biometric signal known as a seismocardiogram (SCG). The SCG signal can be detected and recorded by the accelerometer 205 of the wearable sensor device 200, for example, due to the tight mechano-acoustic coupling of the wearable sensor device 200 to the skin (or other organ) that enables the device to sense mechano-acoustic waveforms that propagate from the internal organs of the body to the surface of the skin. These waveforms are transduced by the onboard accelerometer 205 of the sensor device 200 into electrical signals that the device can measure, record, and store and/or transmit to other devices such as the user device 130 in FIG. 1. In accordance with some embodiments, the SCG waveform can be more reliable than measurement of the ECG for sensors that are attached at points in the body that are relatively far from the heart or chest of the patient.

Alternatively, or in addition, the wearable device 200 can include a discrete temperature sensor, such as the temperature sensor 213, which can be positioned in a different location from the wearable device 200. The wearable device 200 can use the temperature information detected by the temperature sensor 211 and/or the temperature sensor 213 according to various methods and processes. For purposes of convenience, reference is made below to the temperature sensor 211. However, such reference is not limited to apply only to the temperature sensor 211, but applies to any one or more temperature sensors within or connected to the wearable device 200.

The electrical contacts 215 can be formed of conductive material (e.g., copper, silver, gold, aluminum, a hydrogel, conductive polymer, etc.) and provide an interface between the wearable device 200 and the skin of the user 100, for receiving electrical signals (e.g., ECG, EMG, etc.) from the skin. The electrical contacts 215 can include one or more electrical contacts 215, such as two electrical contacts 215, electrically connecting the skin of the user 100 to an amplifier circuit that can be part of an analog front end circuit 216, to amplify and condition electrical signals (e.g., ECG, EMG, etc.). With two electrical contacts 215, one contact can be electrically configured as a positive contact and the other contact can be electrically configured as a negative contact. However, in some aspects, there may be more than two electrical contacts, such as four electrical contacts 215 (e.g., two positive and two negative electrical contacts), six electrical contacts 215, etc. The electrical contacts 215 may also be used as an acoustic contact surface for efficient transmission of acoustic signals to the accelerometer 205.

The optical sensor 217 can measure the photoplethysmography (PPG) signal when placed on the skin's surface, allowing for the monitoring of various biometrics including, but not limited to, heart rate, respiration, and blood oxygen measurements. The optical sensor 217 can include one or more light emitters that can emit red, green, infrared light, or a combination thereof and one or more optical transducers (e.g., photodiode, CCD sensors). Using the one or more optical transducers, the optical sensor 217 can sense the wavelength of the reflected light. In this example, the optical sensor 217 illuminates the skin and the reflected light changes intensity based on the concentration of oxygen in a blood vessel such as an artery or a capillary bed. Thus, a pulse can be detected as a change in the amount of the reflected light due to a change in the concentration of oxygen in a blood vessel and thus the reflected light detected by the optical sensor 217. The system can contain an array of optical sensors in a one-dimensional or two-dimensional grid. In this configuration, the optical sensors can measure reflected light (pulse oxygenation and pulse waveforms) at multiple locations along the vasculature, enabling measurement of time of flight and pulse wave velocity over a given distance (e.g., the separation distance between individual optical sensors.

In addition to the above-described components, the wearable device 200 can include one or more additional components without departing from the spirit and scope of the present disclosure. Such components can include a display (e.g., one or more light-emitting diodes (LEDs), liquid crystal display (LCD), organic light-emitting diode (OLED)), a speaker, a microphone, a vibration motor, a barometer, a light sensor, a photoelectric sensor, or any other sensor for sensing, measuring, or otherwise quantifying parameters and/or characteristics of the body. In other embodiments of the invention, the wearable device 200 can include components for performing one or more additional sensor modalities, such as, but not limited to, hydration level measurements, conductance measurements, and/or pressure measurements. For example, the wearable device 200 can be configured to, or include one or more components that, perform any combination of these different types of sensor measurements, in addition to the accelerometer 205 and temperature sensor 211.

Referring back to the temperature sensor 211, according to some embodiments, the primary purpose of the temperature sensor 211 is for calibrating the accelerometer 205. Accordingly, the temperature sensor 211 does not rely on direct contact to an object to detect the temperature. By way of example, the temperature sensor 211 does not require direct contact to the skin of a user when coupled to the user to determine the skin temperature. For example, the skin temperature affects the temperature information generated by the wearable device 200 without direct contact between the temperature sensor 211 and the skin. Accordingly, the temperature sensor 211 can be fully encapsulated and, therefore, be waterproof for greater durability. The thermal conductivity of the encapsulating material can be selected to control the ability of the temperature sensor 211 to detect the temperature without direct contact.

The wearable device 200 can be constructed of a flexible and/or stretchable printed circuit (e.g., a flex printed circuit board) that can be encapsulated in an elastomer (e.g., silicone, poly urethane, PDMS) that enables the device to stretch and bend. In accordance with some embodiments of the invention, the wearable device 200 can be constructed to have modulus of elasticity (e.g., Young's modulus) similar to the skin of the user or subject. This construction enables the wearable device 200 to be tightly adhered to the skin using a pressure sensitive adhesive such that the sensors in the wearable device are able to detect the slightest motion of the skin as well as the muscles and organ under the skin in the area of the body where the wearable device 200 is attached. This tight coupling can be accomplished using a thin layer (e.g., less than 150 um) of pressure sensitive adhesive and a thin layer (e.g., less than 150 um) of encapsulating material (e.g., silicone). The adhesive and encapsulating materials can be selected to faithfully transmit to the sensors any vibrations or motions from the skin to which it is attached.

The form factor of the wearable device 200 allows positioning and repositioning of the sensor devices at different locations on the body of the user 100 in order to achieve the highest quality of mechano-acoustic data from the accelerometer 205. In this example, the sensor devices 110, 112, 114, and 116 placed on the chest of the user 100 in FIG. 1 can each be configured in electrocardiogram (ECG) mode in order to receive the ECG signal from the user's heart. The ECG signal can be processed by the respective wearable sensor to detect the R-wave portion of the ECG signal and determine a pulse rate from the time-period measured or calculated between the R-waves (e.g., the peaks of the R-wave). Sensor devices 110, 112, 114, and 116 can be a wearable sensor device 200 with the electrical contacts removed (or disabled) that is coupled to the skin (e.g., by an adhesive) and conforms to the body without applying pressure on the arterial wall that would alter the natural motion or flow (and impede the accuracy of the measured motion and vibration signals). This tight coupling also reduces the motion artifacts while enabling high resolution and accurate sensing.

In accordance with some embodiments of the invention, the system shown in FIG. 1 can be used for detection and recording of heartbeat, respiratory, or digestion acoustic data. By designing the accelerometer and encapsulation (to be sub-1 mm and low modulus) to allow intimately coupling with the skin, very fine signals from the chest may be achieved, including coughing, wheezing, and detection of valves opening and closing. Detection of heart murmurs due to improper valve closure and opening may also be detected. The patch may be positioned at a number of locations on the user 100 where vibrations due to pressure waves, sound pressure, and mechano-acoustics are present.

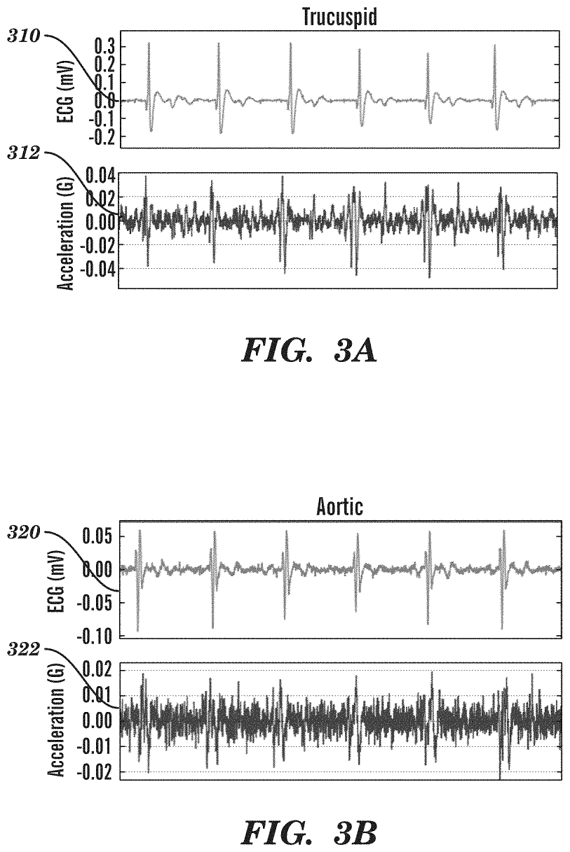

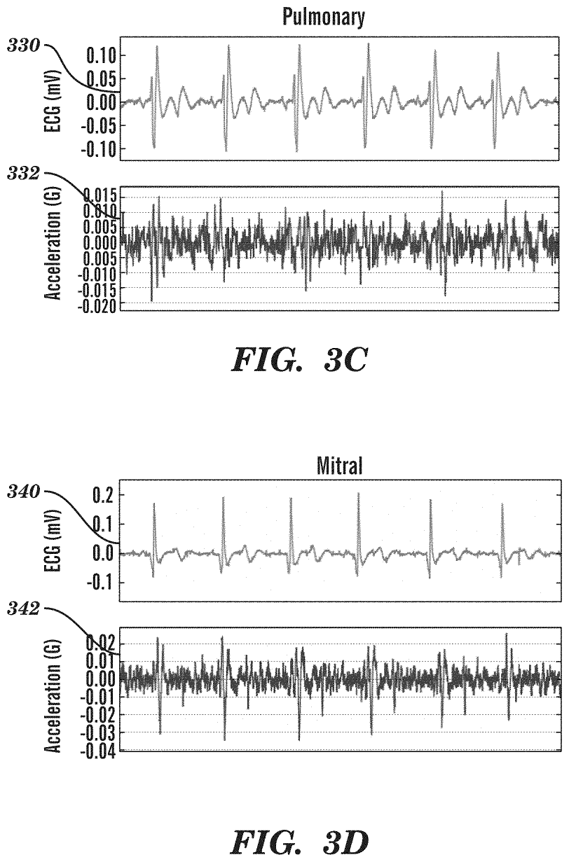

FIGS. 3A-3D are graphs of signal outputs from the wearable sensors 110, 112, 114, and 116 in FIG. 1. FIG. 3A includes an ECG waveform 310 and an accelerometer data output signal 312 taken from the wearable sensor 110 near the aortic valve of the heart. FIG. 3B includes an ECG waveform 320 and an accelerometer data output signal 322 taken from the wearable sensor 112 near the transcuspid valve of the heart. FIG. 3C includes an ECG waveform 330 and an accelerometer data output signal 332 taken from the wearable sensor 114 near the pulmonary valve of the heart. FIG. 3B includes an ECG waveform 340 and an accelerometer data output signal 342 taken from the wearable sensor 116 near the mitral valve of the heart. These outputs show how the system 102 can relate these mechanical vibrations to faster electrical markers driven by cardiac activity or muscle activity. These electrical signals help to verify whether or not the mechano-acoustic signals are physiological or due to motion artifacts. Each location may be used in aggregate with the others to form a cohesive, holistic picture of the cardiac cycle. That is, knowing the ECG and mechano-acoustic signals from each of these four locations and their relative timings can inform an end-user (e.g. physician, cardiologist, patient, etc.) whether the heart valves are operating correctly and are within timing tolerances. If they are not, the heart's pumping efficiency degrades and prevents optimal blood flow through the vasculature. For example, this reduction in efficiency occurs when any of these four valves develops stenosis, or a narrowing of the valve. This can result in the inability of the valve to close properly, promoting backflow of blood within the heart, degrading the pumping mechanics. The mechano-acoustic recording allows one to verify the correct morphology of the waveform; any aberrations from the ideal would indicate an issue with the valve.

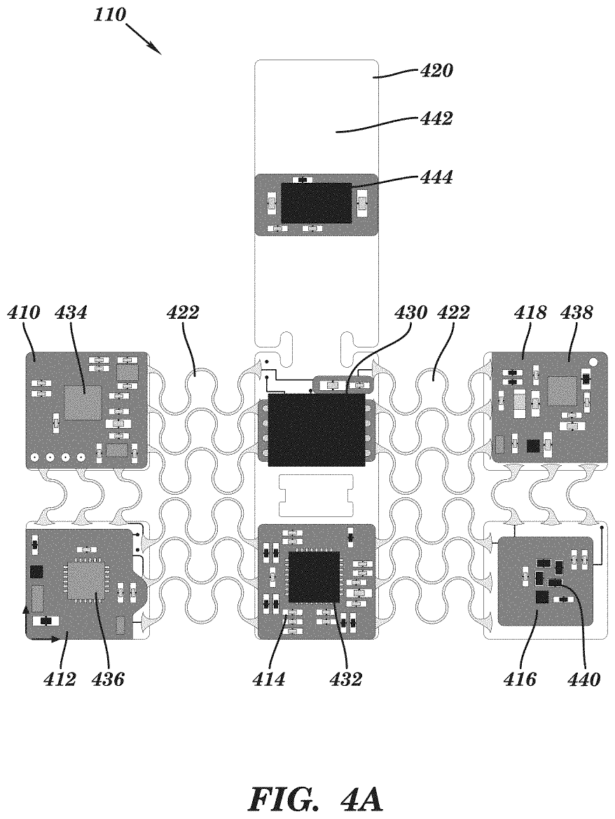

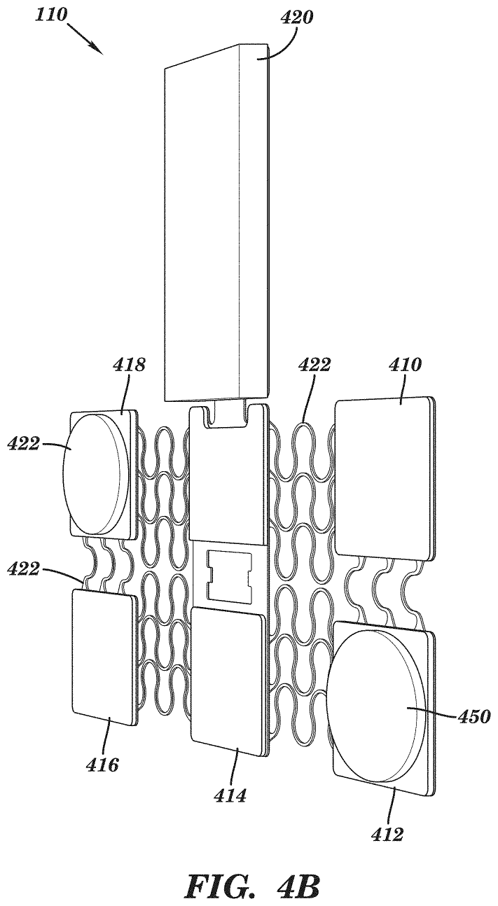

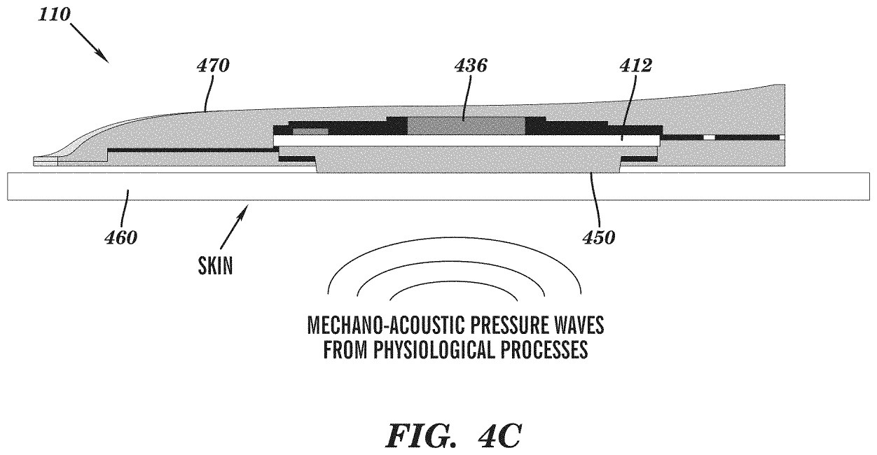

FIG. 4A is a top view of the internal components of the wearable sensor device 110 and FIG. 4B is a bottom perspective view of internal components of the wearable sensor device 110 in FIG. 1. The wearable sensor device 110 includes a number of islands 410, 412, 414, 416, and 418 as well as a battery 420. The islands 410, 412, 414, 416, and 418, and the battery 420 are coupled together by flexible conductive interconnections 422 and are generally positioned in the same horizontal plane. In this example, the flexible conductive interconnections 422 are in a serpentine shape, but other shapes may be used. In this manner, the wearable sensor device 110 can be in conformal contact and flex with movements of a user's skin due to the flexible conductive interconnections 422.

In this example, the overall shape of the sensor device 110 and the plane including the islands 410, 412, 414, 416, and 418 and the battery 420 is a rectangular shape. The battery 420 is centered relative to the rectangular shape and the islands 410 and 412 are arranged on one wing of the sensor device 110 relative to the battery 420. The island 412 is further isolated at a corner of the sensor device 110. Similarly islands 416 and 418 are arranged on an opposite wing of the sensor device 110 opposite the wing including the islands 410 and 412. As will be explained below, the location of the islands 410, 412, 416, and 418 on the wings allows better isolation from dampening effects from the other components of the sensor.

The islands 410, 412, 414, 416, and 418 can be used to support different components (e.g., integrated circuits) on their respective top surfaces as shown in FIG. 4A. In this example, a flash memory chip 430 is mounted on the island 414. A heart rate sensor front end integrated circuit 432 is mounted on the island 410. A microcontroller 434 is mounted on the island 410. A motion sensor 6-axis internal measurement (IMU) integrated circuit 436 that may be used for the accelerometer 205 shown in FIG. 2 is mounted on the island 412. A power management integrated circuit 438 is mounted on the island 414. A series of support components 440 are mounted on the island 416. The memory chip 430 in this example can be a 64 MB memory chip that is part of the memory storage module 203 in FIG. 2. The battery 420 has a flat surface 442 that mounts an optical sensor integrated circuit 444. As will be explained below, the arrangement of the accelerometer components 436 on the island 412 that are on a wing of the sensor device 110 are separated from the other components by the soft and flexible interconnects 422 that isolate the accelerometer from sound and vibration produced by or received by other islands and components of the sensor device 110.

As shown in FIG. 4B, the bottom of the islands 418 and 412 can include respective electrodes 450 and 452 that are in contact with the skin when the wearable sensor 110 is worn by the user. The electrodes 450 and 452 can be electrically connected (e.g., either directly or through an amplifier) to the heart rate sensor integrated circuit 432. Of course, the electrodes 450 and 452 can be included as parts of other islands or in other locations on the islands other than those shown in FIG. 4B. The electrodes 450 and 452 constitute the electrical contacts 215 in FIG. 2. In this example, the battery 420 and the power management integrated circuit 438 constitute the power source 209 in FIG. 2.

In this example, the microcontroller 434 is an onboard nRF52832 system on chip manufactured by Nordic Semiconductor that performs the functions of the processor 201 and transceiver 207 in FIG. 2. In this example, the microcontroller 434 is an ultra-low power multiprotocol system on chip suited for Bluetooth® low energy communication, ANT and 2.4 GHz ultra low-power wireless applications. The system on chip includes a CPU that supports DSP instructions, a Floating Point Unit (FPU), single-cycle multiply and accumulate, and hardware divide for energy-efficient process of computationally complex operations. The microcontroller 434 includes an embedded transceiver that supports Bluetooth low energy, ANT and proprietary 2.4 GHz protocol stack. The microcontroller also includes a multiprotocol radio that includes DMA for direct memory access during packet send and retrieve.

In this example, the heart rate sensor front end integrated circuit 432 is an ADS1191 chip manufactured by Texas Instruments and can be an integrated part of the processor 201 in FIG. 2. The front end integrated circuit 432 in this example is a multichannel, simultaneous sampling, 16-bit, delta-sigma analog-to-digital converter (ADCs) with a built-in programmable gain amplifier (PGA), internal reference, and an onboard oscillator. The front end integrated circuit 432 has a flexible input multiplexer per channel that can be independently connected to the internally-generated signals for test, temperature, and lead-off detection. The heart rate sensor front end integrated circuit 432 makes electrical contact with the subject's skin via electrodes 450 and 452 on the skin-facing side of the device as shown in FIG. 4B.

The optical sensor integrated circuit 440 is a MAX30101 chip manufactured by Maxim Integrated and serves as the optical sensor 217 in FIG. 2. In this example, the optical sensor integrated circuit 440 includes internal LEDs, photodetectors, optical elements, and low-noise electronics with ambient light rejection. The sensor includes a reflective LED based heart-rate monitor and a pulse oximeter sensor.

FIG. 4C is a side view of the wearable sensor 110 showing the island 412 and electrode 450 in contact with the skin 460 of the user. The island 412 and the other internal components are encapsulated in an encapsulation material 470 that is flexible to allow the wearable sensor device 110 to conform with the distortions in the skin 460. In this example, the encapsulation material 470 is an elastomer (e.g., silicone, poly urethane, PDMS), but any sufficiently protective and flexible material may be used. As shown in FIG. 4C, the encapsulation material 470 is not formed over the electrode 450 to allow direct contact with the skin and thus provide the most highly conductive path for transmission of the mechano-acoustic signal to the mechano-acoustic sensor (e.g., the accelerometer or IMU). Because of the direct contact with the skin provided by the electrode 450, this configuration provides a highly effective, low distortion mechano-coustic path from the skin to the integrated circuit 436 that is part of the accelerometer.

The system 102 in FIG. 1 functions as a digital stethoscope that uses an accelerometer with tight mechanical coupling to the user's skin. Thus, the example wearable sensor device 110 (as well as the other wearable sensor devices 112, 114, and 116) shown in FIGS. 4A-4C has salient features that allow for this high level of coupling.

The accelerometer integrated circuit 436 is placed on an isolated island such as the corner island 412. This placement benefits from an extra horizontal column of the serpentine interconnections 422, which, like a spring, decouples the mass of the corner island 412 from the rest of the device 110. Alternatively, the flexible interconnections 422 around the accelerometer sensor island may be made sufficiently soft to further decouple the accelerometer sensor from the rest of the sensor device 110 and thus isolating the accelerometer sensor and enabling the use of a highly sensitive sensor to detect lower levels of vibrations. For example, these interconnections can be made of thinner metal trace materials relative to the other flexible interconnections and/or use a softer metal material. Trace material and physical dimensions are useful factors in minimizing stiffness and maximizing reliability. Certain materials, for example rolled-annealed copper, have mechanical properties that are amenable to bending and stretching. This is due to the structure of the molecules which align like wood grains that allows for easy bending and flexing while maintaining high mechanical reliability along the direction of the grain. Additionally, an interconnection may have an overall trace thickness and width of 12 μm and 75 μm, respectively. When these dimensions are used in serpentines, they minimize stiffness and allow islands to decouple mechanically. As a point of comparison, if the thickness were doubled to 24 μm, the bending stiffness would increase by a factor of 8 and the stretching stiffness would increase by a factor of 2. This occurs since the bending and stretching area moments of inertia for a trace is related by the equation bh³/12 for the former and hb³/12 for the latter, where b and h are trace width and thickness, respectively. These moments of inertia are directly proportional to stiffness. With a high area moment of inertia, a trace will have a high amount of stiffness. Thinner trace dimensions help ensure less stiffness and a softer serpentine. Both the features of an additional horizontal column and a softer serpentine can be combined. This ensures that any vibration picked up by the accelerometer integrated circuit 436 at the skin surface comes from an internal organ of the body and not from the mechanical movements of the other islands 410 and 414, (i.e., motion artifacts). In this example, the electrode 450 is in direct contact with the skin 460 and therefore directly transmits the mechano-acoustic signals received from the surface of the skin to the accelerometer integrated circuit 436.

The design on the wearable device 110 having the battery centrally located with the acoustic sensor isolated on the wings prevents lift-off of the device on the skin. Since the battery 420 is the most massive component in the device, it could cause the device to peel if positioned on an edge of the device. By being in the middle of the device, the battery 420 has less effect on overall device lift-off, mitigating any unintended mechanical movements of the device relative to the skin that may transmit motion or acoustic artifacts to the accelerometer integrated circuit 436. The location of the island 412 in a corner of the rectangular plane relative to the middle position of the battery 420 thus isolates the island 412 from mechano-acoustic signals of the other islands 410, 414, 416, and 418.

With the onboard heart rate sensor integrated circuit 432, the wearable sensor device 110 can reduce false-positives in the stethoscope recording function of the system 102 by correlating mechano-acoustic signals from the accelerometer integrated circuit 436 with their corresponding electrical signal from the heart rate sensor integrated circuit 432. A heartbeat can be successfully identified via the accelerometer integrated circuit 436 if there is a valid ECG signal that precedes it since bioelectrical signals are present before the accompanying mechanical one. This may be accomplished by the use of algorithms that can appropriately detect the R-wave component of an ECG, signifying to the user that an ECG pulse is present. The ECG pulse verifies that a valid cardiac cycle has taken place. Given this event in normal subjects, an accompanying mechano-acoustic signal must be generated, since a valid cardiac-electrical cycle cannot occur without cardiac-mechanical activity. Once this determination is made, any mechano-acoustic waveform that matches one of the signal morphologies shown in FIGS. 3A-3D may be classified as valid. The waveform morphology may be matched by a correlation filter in either the time or frequency domains. By combining the signals of two or more sensors, the digital stethoscope function of the system 102 can reduce sensitivity to noise and motion artifacts, increasing the overall signal quality (e.g., signal-to-noise ratio for the mechano-acoustic signals of interest).

The thin encapsulation layer 470 promotes tight mechanical coupling between the accelerometer integrated circuit 436 and the user's skin. This ultimately allows for efficient transduction of mechano-acoustic energy (induced by physiological processes) to the accelerometer integrated circuit 436.

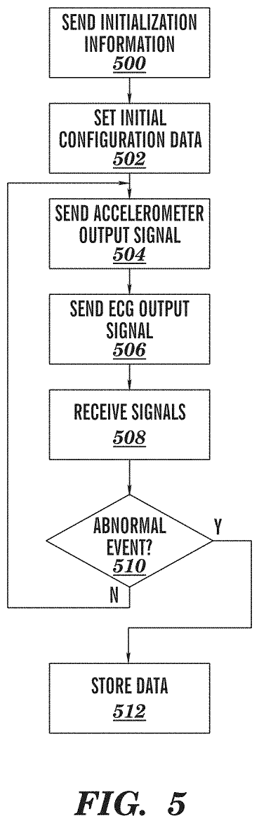

FIG. 5 is a flow diagram of the process of collecting mechano-acoustic data and determining an abnormal heart function in the system 102 shown in FIG. 1. Handshaking is performed between the user device 130 and the sensor devices 110, 112, 114, and 116 (500). The handshaking involves sending identification information for the sensor devices 110, 112, 114, and 116 and respective MAC addresses to the user device 130. The user device 130 sets initial configuration data such as the location of the sensor devices 110, 112, 114, and 116 on the body, the sampling rate and applicable storage parameters (502).

The sensor devices 110, 112, 114, and 116 continuously (or periodically) send an output accelerometer signal to the user device 130 that can include one or more samples (e.g., 2, 3, 4, 5, 10, 20, or more samples) associated with a particular timestamp (504). As explained above, each of the sensor devices 110, 112, 114, and 116 gathers acoustic data from different valves in the heart reflecting circulation of blood in the circulatory system. In this example, the sensor devices 110, 112, 114, and 116 can continuously (or periodically) send the output of the ECG signal received from the electrical contacts 215 in FIG. 2 to the user device 130 in order to confirm the data relating to circulation of blood from the heartbeat (506). The output of the ECG signal can include one or more samples (e.g., 2, 3, 4, 5, 10, 20, or more samples) associated with a particular timestamp. The ECG signal is optional for the application relating to heartbeat data. For other mechano-acoustic measurements, the ECG signal gathering step may be eliminated or other types of data may be sensed to assist in confirming the mechano-acoustic measurements. For example, a system may use the optical sensor 217 to determine if a mechano-acoustic event is valid. This can be accomplished by time-aligning an appropriate optical sensor waveform (PPG) event with a corresponding mechano-acoustic event. Using this sensor, the PPG waveform can be correlated to the mechano-acoustic waveform in a process similar to that described above.

The user device 130 receives the accelerometer output waveform signal and the ECG output waveform signals from each of the sensor devices 110, 112, 114, and 116 (508). The user device 130 determines whether there is an abnormal event such as an irregular heartbeat based on the analysis of the received data (510). Since each sensor 110, 112, 114, and 116 is monitoring a different part of the heart, the source of the abnormal event may be isolated. If there is no abnormal event, the user device 130 returns to receiving the output signals (508). If an interruption is detected, the user device 130 stores the data from the waveform signals in memory (512). As explained above, the stored data may be used as part of an input to an LVAD implantable device or pump that changes its routine based on the mechanoacoustic input signals.

Alternatively, the timestamp data and respective signals may be transmitted to the cloud server 142 and some or all of the above operations may be performed by the cloud server 142. Alternatively, the sensor device 110 or the sensor device 112 may store the waveform data and transmit the stored data periodically to the user device 130 for analysis of sleep interruption or abnormal patterns at a delayed time.

Thus, a single device such as the wearable sensor device 110 constitutes an epidermal device that can capture both ECG and mechano-acoustic vibrations of the chest in a way that allows proper characterization of electrical and mechanical waves propagating through the soft tissues of the human body for heart-based monitoring. As explained above, the wearable sensor has an accelerometer very closely coupled to skin surface. Of course, using multiple sensors will allow further isolation of events on different parts monitored by the corresponding sensors. As shown in FIGS. 4A-4C, the accelerometer is encapsulated with very soft, thin layer of silicone, which allows for direct coupling of mechanical vibrations and wave propagation along the chest cavity to the accelerometer sensor. This direct coupling allows the epidermal accelerometer in the wearable sensor device 110 to become a wearable electronic stethoscope. There are design variants to optimize coupling of the accelerometer with skin even further to enhance signal quality even further. For example, the accelerometer could be mounted on the undersurface of the wearable sensor device (facing the skin surface). This design variant would in turn couple the accelerometer directly with the surface of skin (without having the flexboard barrier).

The digital stethoscope function of the system 102 may have numerous uses as explained above in relation to heart, respiratory, and digestive monitoring. For example, heart murmurs may be detected from wearable sensors such as the sensors 110, 112, 114, and 116 that are attached in proximity to the heart of the user. A heart murmur is detected by blood rushing quickly through the valves that creates a unique acoustic signal. Another example is detection of a ventricular defect that is detected via the presence of a third heart sound (S3 or ventricular gallop) that is like a low frequency vibration.

As explained above, some or all of the wearable sensors 110, 112, 114, and 116 may be used for respiratory monitoring. In such monitoring, sensors would be attached on the user 100 at the lower part of the neck, where the clavicles and lower neck meet. In addition, any location on the chest would suffice for detecting respiration. The user device 130 would be configured to detect data relating to respiration. Such data may include bronchial breath sounds detected from within the tracheobronchial tree or vesicular breath sounds heard over the lung tissue.

Abnormal breath sounds include wheezing, stridor, rhonchi, and rales sounds. Further, the absence of breathing sounds may indicate air or fluid around the lungs, thickness around the chest wall, or airflow that is slowed down or over inflation to the lungs. Wheezing sounds like a high pitched sound when the person exhales, and sometimes when they inhale may indicate asthma. Stridor sounds like high-pitched musical breathing, similar to wheezing, heard most often when the patient inhales. Stridor is caused by a blockage in the back of the throat. Rhonchi sounds like snoring and is a result of the air following a “rough” path through the lungs or because airflow is blocked. Rales sounds like popping bubble wrap or rattling in the lungs and may indicate respiratory disease.

The system 102 can also be used to monitor digestive functions. In such monitoring, sensors would be attached on the user 100 at lower trunk area where the stomach and intestines are located. The user device 130 would be configured to detect data relating to digestion. Bowel sounds may be compared to normal functioning bowel sounds to determine the presence of abnormalities. The absence of any bowel sounds may indicate something is blocked in the patient's stomach or constipation. Over the course of monitoring the digestive system over a period of time, if the patient has hyperactive bowel sounds followed by a lack of bowel sounds, a rupture or necrosis of the bowel tissue could be detected. Very high-pitched bowel sounds, can indicate that there is an obstruction in the patient's bowels. Slow bowel sounds may be caused by prescription drugs, spinal anesthesia, infection, trauma, abdominal surgery, or overexpansion of the bowel. Fast or hyperactive bowel sounds can be caused by Crohn's disease, a gastrointestinal bleed, food allergies, diarrhea, infection, and ulcerative colitis.

The blood flow in other parts of a patient's body may be monitored by taking mechano-acoustic data. For example, a bruit may be detected in the renal arteries, iliac arteries, and the femoral arties by detecting a whooshing sound that indicates that the artery is narrowed.

There are several commercial applications ranging from in-home wearable stethoscopes for monitoring valve opening and closure post- or pre-operatively. If patients are experiencing heart murmurs, a wearable stethoscope system could help detect murmurs during sleep or during rest. This monitoring system could be a companion device with artificial valve implants to monitor performance over time post-procedure. Ventricular assist devices (VAD) could benefit from having these non-invasive wearables, which could track vibrations caused by the VAD pump. These vibrations could indicate potential failure modes where blood flow through the VAD may be reduced or obstructed due to a latent pathology. Once detected by the digital stethoscope, these vibrations could motivate a clinician to run a more complete battery of tests to determine the efficacy of the VAD and/or overall heart health.

In some embodiments, the aforementioned methods include at least those steps enumerated above. It is also within the scope and spirit of the present disclosure to omit steps, include additional steps, and/or modify the order of steps presented herein. It should be further noted that each of the foregoing methods can be representative of a single sequence of related steps; however, it is expected that each of these methods will be practiced in a systematic and repetitive manner.

While particular embodiments and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of the invention as defined in the appended claims.

1. A sensor system for sensing sound associated with a bodily function of a user, the system comprising:
a wearable sensor including a planar mechano-acoustic conductor in direct contact with the skin of the user to measure mechano-acoustic vibration signals generated from a bodily function and generate a vibration waveform; and
a controller that receives the mechano-acoustic vibration waveform from the wearable sensor to determine a measurement of the bodily function.

2. The sensor system of claim 1, wherein the bodily function is one of a heart function, a respiratory function or a digestive function.

3. The sensor system of claim 2, further comprising a heartbeat sensor in contact with the skin of a user to measure a heartbeat waveform, wherein the controller receives the heartbeat waveform from the heartbeat sensor and determines a heartbeat measurement from the mechano-acoustic vibration waveform and the heartbeat waveform.

4. The sensor system of claim 3, wherein the heartbeat sensor is one of an ECG sensor, a SCG sensor or a PPG sensor.

5. The sensor system of claim 3, wherein the controller is operative to detect an abnormality in heart function based on the mechano-acoustic vibration waveform.

6. The sensor system of claim 1, wherein the wearable sensor includes an accelerometer to measure the mechano-acoustic vibration signals.

7. The sensor system of claim 6, wherein another accelerometer sensor is attached in another area on the body to sense an accelerometer waveform based on an acoustic signal from the another area of the user.

8. The sensor system of claim 1, further comprising an external device in communication with the controller.

9. The sensor system of claim 1, further comprising a memory for storing the mechano-acoustic vibration waveform.

10. The sensor system of claim 1, further comprising a transceiver to transmit the mechano-acoustic vibration waveform.

11. The sensor system of claim 10, further comprising a user device in communication with the transceiver to receive the mechano-acoustic vibration waveform, the user device operative to determine an abnormality in the monitored bodily function.

12. The sensor system of claim 1, wherein the wearable sensor has a plurality of islands in a planar configuration, wherein the mechano-acoustic conductor is attached to an island isolated from the other islands in the plurality of islands via stretchable interconnects.

13. The sensor system of claim 11, wherein the plurality of islands is encapsulated in an elastomer material.

14. The sensor system of claim 11, wherein the wearable sensor has a rectangular planar shape, wherein the island attached to the mechano-acoustic conductor is in one corner of the rectangular planar shape.

15. The sensor system of claim 14, wherein the sensor includes is an accelerometer integrated circuit and wherein the island attached to the mechano-acoustic conductor includes a first surface mounting the accelerometer integrated circuit and an opposite second surface supporting the planar mechano-acoustic conductor.

16. The sensor system of claim 14, wherein the planar mechano-acoustic conductor also functions as an ECG electrode.

17. A wearable sensor for detecting a mechano-accoustical signal from a user, the sensor comprising:
a rectangular planar body composed of encapsulation material;
a first island in the middle of the rectangular planar body; and
a second island including an accelerometer, the second island being isolated from the first island using flexible interconnections to buffer vibrations, wherein the second island is located in proximity to a corner of the rectangular planar body.

18. The sensor of claim 17, wherein the first island includes a battery.

19. The sensor of claim 17, wherein the second island has a top surface and an opposite bottom surface, wherein the bottom surface includes a contact in direct contact with the skin and the top surface holds the accelerometer.

20. The sensor of claim 17, wherein the first island includes a heart rate monitor and the contact is an electrode coupled to the heart rate monitor.

21. A method of detecting an acoustic signal from a user, the method comprising:
attaching a wearable sensor including a planar mechano-acoustic conductor in direct contact with the skin of the user to measure mechano-acoustic vibration signals generated from a bodily function and generate a vibration waveform; and
determining a measurement of the bodily function from the mechano-acoustic vibration waveform via a controller.

22-36. (canceled)
